(19) United States
(12) Patent Application Publication
(10) Pub. No.: US 2025/0276951 A1
(43) Pub. Date: Sep. 4, 2025

(54) PROCESSES FOR MAKING VINYL ACETATE FROM ETHYLENE GLYCOL DIACETATE

(71) Applicant: New Product Innovation, LLC, Johnston, IA (US)

(72) Inventors: David James Schreck, Lake City, MN (US); Norman L. Balmer, Marana, AZ (US)

(73) Assignee: New Product Innovation, LLC, Johnston, IA (US)

(21) Appl. No.: 19/070,184

(22) Filed: Mar. 4, 2025

Related U.S. Application Data
(60) Provisional application No. 63/561,119, filed on Mar. 4, 2024.

(51) Int. Cl. C07C 67/297

(52) U.S. Cl. CPC C07C 67/297

(57) ABSTRACT

The various embodiments disclosed herein relate to a process for producing vinyl acetate monomer (VAM) from ethylene glycol diacetate (GDA) by thermal cracking in the presence of a substantially non-reactive liquid. The liquid phase enhances heat transfer, improves selectivity, and reduces fouling. The process operates in a continuous or discontinuous phase, optimizing efficiency and enabling higher yields of VAM with improved reactor performance.

### CROSS-REFERENCES & RELATED APPLICATIONS

This application claims priority to U.S. Provisional Application No. 63/561,119 filed Mar. 4, 2024 and entitled “PROCESSES FOR MAKING VINYL ACETATE FROM ETHYLENE GLYCOL DIACETATE,” which is hereby incorporated by reference in its entirety under 35 U.S.C. § 119 (e).

### BACKGROUND

Vinyl acetate monomer (VAM) is an important commodity chemical. Numerous processes exist for making VAM. An interest exists in using sustainable feedstocks rather than fossil-based feedstocks to make commodity chemicals, including VAM. Commercial acceptance of commodity chemicals made using sustainable feedstocks will, in part, depend on the cost of their production.

Vinyl acetate is typically made by the catalytic reaction of ethylene, acetic acid and oxygen. Some industrial production uses the hydroesterification of acetic acid and acetylene. A proposed route is the thermal or catalytic cracking of 1,1-diacetoxyethane. See, for instance, U.S. Pat. No. 2,425,389 and European Patent Application Publication No. 0 048 173 A1. The high costs of producing 1,1-diacetoxyethane have not led to this proposed route achieving commercial viability for making VAM.

Vinyl acetate monomer can also be made from ethylene glycol diacetate. U.S. Pat. Nos. 2,251,983; 3,689,535; 3,787,485 and 3,804,887 disclose the thermal cracking of ethylene glycol diacetate to vinyl acetate. The thermal cracking of ethylene glycol diacetate has also not seen commercial viability.

Nevertheless, providing an economically attractive route for making vinyl acetate monomer from ethylene glycol diacetate is desired as the ethylene glycol diacetate can be obtained from renewable as well as fossil-based feedstocks. Making vinyl acetate monomer from sustainable resources can proceed through several routes. For instance, ethylene glycol diacetate can be made by the esterification of monoethylene glycol. The conventional process for making monoethylene glycol is by cracking fossil-based feedstock to produce ethylene, partially oxidizing the ethylene-to-ethylene oxide and then hydrolyzing ethylene oxide to monoethylene glycol. The conventional process could use ethylene that is derived from sustainable feedstocks, e.g., ethylene made by the dehydration of ethanol derived from the fermentation of carbohydrate. Other processes exist for making monoethylene glycol from sustainable feedstocks. For instance, sustainable feedstock can be converted to syngas and monoethylene glycol can be made by the glycol oxalate process such as disclosed in U.S. Pat. No. 4,453,026. Also, recent efforts have been devoted to converting carbohydrates to monoethylene glycol by retro aldol conversion of the carbohydrate to, among others, glycol aldehyde, and hydrogenation to monoethylene glycol. See, for instance, U.S. Pat. No. 9,783,472.

The commercial viability of processes for making vinyl acetate monomer from ethylene glycol diacetate, whether from fossil sources or sustainable resources, will depend upon, among other things, the efficiencies of converting ethylene glycol diacetate to vinyl acetate monomer.

### BRIEF SUMMARY

This invention pertains to processes for making vinyl acetate monomer from ethylene glycol diacetate wherein ethylene glycol diacetate is contacted with a continuous or discontinuous liquid phase of a substantially non-reactive component, which component is at a temperature sufficient to convert ethylene glycol diacetate to vinyl acetate monomer and acetic acid (“cracking temperature”). By this invention it is believed that a number of advantages can be achieved. Most notably, the contact of ethylene glycol diacetate and vinyl acetate monomer at cracking temperatures with metal surfaces that can promote undesired reactions or become fouled, can be maintained at a low incidence. Thus, enhanced selectivities to vinyl acetate monomer and reduced reactor maintenance costs to address fouling, can be achieved. Moreover, as the conversion of ethylene glycol diacetate to vinyl acetate monomer is endothermic, the continuous or discontinuous liquid phase provides heat proximate to the sites of the cracking thereby enhancing conversion rates. The ability to provide heat proximate to the sites of the cracking liquid phase enables a relatively uniform temperature profile in the reaction zone as compared to conventional thermal cracking processes where the heat is only provided at the walls of the reaction zone. Thus, the operator has better control of the process for optimization of production of vinyl acetate monomer. In some instances, enhanced net production of vinyl acetate monomer per unit of ethylene glycol acetate fed to the reaction zone can be achieved as compared to thermal cracking where the heat is provided by the walls of the reactor all other conditions being substantially the same.

In the broad aspects, the processes of this invention comprise: continuously supplying ethylene glycol diacetate to a reaction zone; contacting in the reaction zone the ethylene glycol diacetate with a substantially non-reactive liquid, said liquid being (i) at a temperature higher than the boiling point at the pressure in the reaction zone of the ethylene glycol diacetate supplied such that a mixed liquid-vapor phase exists in the reaction zone, and (ii) at a temperature sufficient to convert at least a portion of the ethylene glycol diacetate to vinyl acetate monomer and acetic acid and provide a product gas comprising vinyl acetate monomer, acetic acid and unreacted ethylene glycol diacetate; and continuously withdrawing said product gas from the reaction zone.

The ethylene glycol diacetate can be supplied to the reaction zone as a liquid or a vapor. Where supplied as a liquid, the ethylene glycol diacetate vaporizes. Thus, in either event a vapor phase and a liquid phase will exist in the reaction zone. In some instances, the ethylene glycol diacetate is preheated to a temperature between about 250° C. and 450° C., say, 350° C. to 425° C., which is below a temperature where significant cracking occurs, and the ethylene glycol diacetate is already in the vapor phase prior to contact with the liquid. Thus, the liquid need not provide the latent heat of vaporization and only needs to provide the sensible heat required to bring the ethylene glycol diacetate to reaction temperature.

Often the liquid is at a temperature within the range of about 400° C. to 570° C., say, 450° C. to 550° C. The amount of liquid used, in addition to providing any latent heat for vaporization or sensible heat for raising the temperature of the ethylene glycol diacetate, is sufficient to attenuate the loss of heat in the region proximate to a molecule of ethylene glycol diacetate undergoing cracking to generate vinyl acetate and acetic acid. The liquid phase can be the continuous phase or the discontinuous phase in the reaction zone. Where the liquid phase is the discontinuous phase, it is preferred that the liquid phase be in a relatively uniform distribution in at least a portion of the cross-section of the reaction zone perpendicular to the direction of flow of the vaporous phase passing through the reactor. Operations using the liquid as the continuous phase are typically preferred. Often the design of the reaction zone is such that the ethylene glycol diacetate is passed to a lower portion of the liquid phase as small bubbles, say, less than 2 centimeters in diameter, preferably less than 5 millimeters, and sometimes less than 20 microns in diameter.

Most frequently only a portion of the ethylene glycol diacetate fed to the reaction zone is converted, say, less than 75 percent, and often between about 15 and 50 percent, is converted. In such cases, the product gas withdrawn from the reaction zone would contain unreacted ethylene glycol diacetate. The product gas can be cooled to a temperature to condense at least a portion of the unreacted ethylene glycol diacetate but maintain the vinyl acetate monomer in the vaporous phase, the condensate separated from the vaporous phase and at least a portion of the condensate is recycled to reaction zone.

### BRIEF DESCRIPTION OF THE DRAWINGS

FIGS. 1 and 2 are schematic depictions of an apparatus that can be used in the processes of the claimed processes.

FIG. 3 is a schematic depiction of an injector that can be used to contact the non-reactive liquid with ethylene glycol diacetate which is either in the liquid phase or vaporous phase.

### DETAILED DESCRIPTION

All patents, published patent applications, and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3, 0.63, 0.815 and so forth) are included as are all intermediate ranges (e.g., 0.2-0.5, 0.54-0.913, and so forth).

The use of the terms “a” and “an” is intended to include one or more of the elements described.

Admixing or admixed means the formation of a physical combination of two or more elements which may have a uniform or non-uniform composition throughout and includes, but is not limited to, solid mixtures, solutions and suspensions.

Ethylene glycol acetate is also known as 1,2-diacetoxyethane and ethylene glycol diacetate is abbreviated herein as GDA.

The half acetate ester of ethylene glycol is also known as 2-hydroxyethyl acetate and is abbreviated herein as HEA.

Substantially non-reactive means that under the reaction conditions including time, temperature and presence of catalyst and other adjuvants, less than 1 percent of the moiety would be reacted.

Sustainable resources are plants and animals, including, but not limited to, waste products from plants and animals, and sustainable feedstocks are derived from sustainable resources. Sustainable resources also include carbon dioxide used as a feedstock in processes to make monoethylene glycol, whether that carbon dioxide is captured from direct air capture or from emissions from facilities that emit carbon dioxide, including, but not limited to, incineration, power generation, fermentations, and chemical and other industrial processes.

According to various implementations, ethylene glycol diacetate in the vaporous phase can be converted to vinyl acetate monomer and acetic acid in the presence of a substantially non-reactive liquid. In these implementations, the liquid has a boiling point under the pressure in the reaction zone equal to, or preferably higher; in one example, at least about 10° C. higher than the bulk temperature in the reaction zone. Examples of liquids include higher molecular weight hydrocarbons which may be aliphatic or aromatic, with 30 or more carbons. Useful liquids include paraffins of 36 or more carbons, especially normal paraffins of 36 to 50 carbons. Liquids in which acetic acid has limited solubility, such as higher molecular weight paraffins, are particularly advantageous.

In some implementations, the amount of liquid used is sufficient to attenuate the loss of heat proximate to a site where ethylene glycol diacetate is being cracked to vinyl acetate and acetic acid. A number of factors may be taken into consideration when determining the amount of liquid to be used. In implementations where the liquid is the continuous phase, the primary variables are the temperature of the liquid and the desired degree of conversion of ethylene glycol diacetate. In one exemplary implementation, at lower liquid temperatures, the rate of conversion may be slower, and thus for increased conversion, more liquid would be used. Also, the geometric configuration of and flow path of bubbles of ethylene glycol diacetate of the reaction zone may be a factor. In some implementations where the liquid is a discontinuous phase, additional factors may need to be taken into account such as the extent that the walls of the reaction zone provide heating or cooling and the duration of contact between the liquid and vaporous phases. As would be understood, the rate of supply of the ethylene glycol diacetate to the reaction zone may be such that at least 2, optionally at least 5, grams of liquid phase are present in the reaction zone per gram of ethylene glycol diacetate fed per second to the reaction zone. In some implementations, especially where the liquid phase is the continuous phase, at least 50, such as, 100 to 5000, grams of liquid phase are present in the reaction zone per gram of ethylene glycol diacetate fed per second to the reaction zone.

The ethylene glycol diacetate fed to the reaction zone can be about all ethylene glycol diacetate or be in combination with one or more components, for instance, propylene glycol diacetate and/or 1,2-diacetoxybutane. Other components can include, but are not limited to, diluents such as inert gases and hydrocarbons such as methane and ethane. Cracking can provide acetic acid and vinyl acetate and, if propylene glycol diacetate is in the feedstock, allyl acetate, and if 1,2-diacetoxybutane is in the feedstock, acetoxybutene such as one or more of 1-acetoxy-2-butene, 2-acetoxy-1-butene, and 2-acetoxy-2-butene. A vaporous phase comprising these components may be withdrawn from the reaction zone and subjected to distillation to provide a vinyl acetate rich stream. Preferably the vinyl acetate rich stream contains less than 0.1, most preferably less than 0.01, mass percent allyl acetate and less than 50 parts per million by mass of any compound containing four or more carbons bonded in series.

The temperatures in the reaction zone may be in the range of about 350° C. to 750° C., say, between about 450° C. to about 550° C., are often employed, with higher temperatures being used in combination with higher space velocities than can be used at lower temperatures. Residence times at higher temperatures, e.g., above about 450° C., may be limited to attenuate adverse reactions such as the generation of ethylene from the vinyl acetate. In some implementations, the time that the vaporous phase is at a temperature of above about 350° C. is less than about 200 seconds, and frequently less than about 50 seconds, or even less than about 10 seconds. In some implementations with temperatures above about 450° C. are used, the duration that the vaporous phase is at or above about 450° C. is often less than about 10 seconds, and sometimes less than about 5 seconds. Dilution of the ethylene glycol diacetate in the vaporous phase with an inert, e.g., nitrogen, methane, and ethane, where the cracking is in the vaporous phase, can assist in achieving short residence times. The reaction zone pressure can be in the range of below atmospheric to 5000 kPa or more, and often in the range of about 50 to 2500 kPa absolute. Higher pressures serve to increase the boiling point of the non-reactive liquid thereby enabling the use of liquids that would otherwise boil and serve to reduce the fraction of liquid in the vapor phase.

It would be appreciated that with very short residence times at temperature, only a portion of the ethylene glycol diacetate would be converted. In some implementations, the conversion of ethylene glycol diacetate is between about 10 percent and about 75 percent, say between about 15 percent and about 50 percent. Generally, at the lower conversions, the selectivity to vinyl acetate is enhanced, all else being substantially constant. Preferably, the thermal cracking temperature, space velocity and time at temperatures above 350° C., provide a selectivity of ethylene glycol diacetate converted to vinyl acetate of at least about 60, and sometimes at least about 70, percent. The ethylene glycol diacetate not converted can be recycled, after removal of vinyl acetate, to the reaction zone.

Any suitable reactor or series of reactors can be employed in conducting the cracking, and the process can be batch, semi-continuous or, preferably, continuous. A reactor can be one or more vessels in series or in parallel and a vessel can contain one or more zones. A reactor can be of any suitable design for continuous operation including, but not limited to, tanks and pipe or tubular reactor and can have, if desired, fluid mixing capabilities. Types of reactors include, but are not limited to, tubular reactors, stirred tank reactors, trickle-bed reactors, falling film reactors, bubble column, sieve trayed column, valve trayed column, packed and loop reactors. In some instances, the cracking may be conducted in whole or part in a reactive distillation apparatus used to make the ethylene glycol diacetate. In some instances, the cracking may be conducted in a separate reactive distillation apparatus from that used to make the ethylene glycol diacetate. It should be understood that the cracking may be conducted in one or more zones, each being at the same or being at different cracking conditions, including, but not limited to, the same or different residence times, pressures, and temperatures.

The heavies from the cracking contain unreacted ethylene glycol diacetate and other high boiling esters, if present, such as propylene glycol diacetate and 1,2-acetoxybutane. Often the distillation is designed to obtain an off-take of ethylene glycol diacetate and other diacetates, if present, for recycle to the reaction zone. The diacetate fraction can be withdrawn from the distillation as a liquid phase or as a vapor phase.

### DRAWINGS

Reference is made to the drawings which are provided to facilitate the understanding of the instantly disclosed process but are not intended to be in limitation of the disclosed process. The drawings omit ancillary unit operations and omit minor equipment such as pumps, heat exchangers, valves, instruments and other devices and unit operations the placement of which, and operation thereof, are well known to those practiced in chemical engineering.

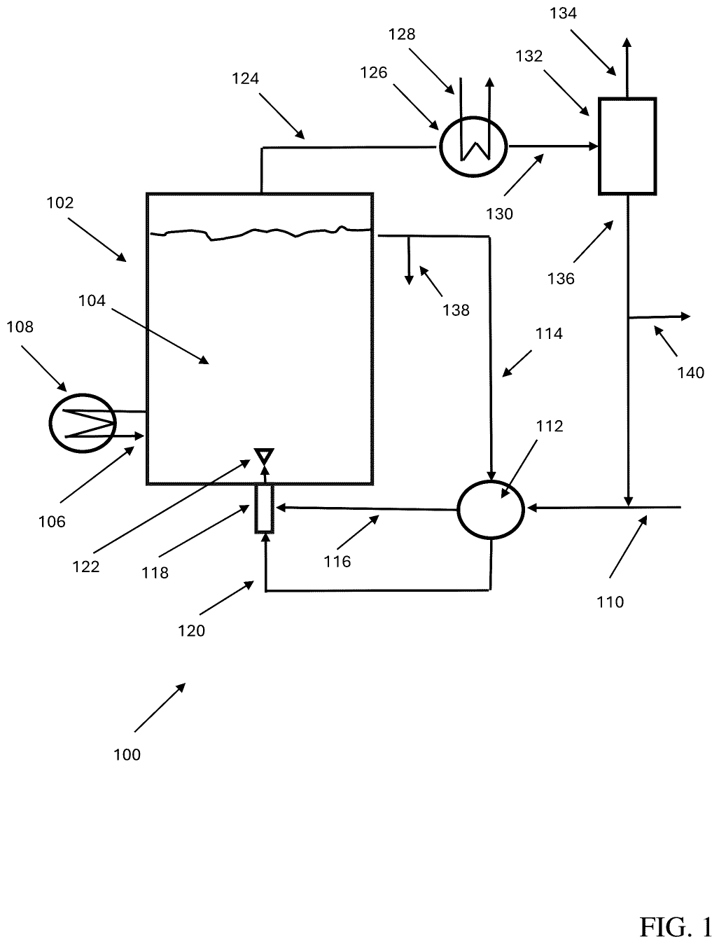

FIG. 1 is a schematic depiction of an apparatus generally designated as 100 suitable for practicing the described processes where the non-reactive liquid is the continuous phase in the reaction zone. The apparatus comprises reactor 102 which for the purposes of discussion can be an unstirred tank containing a liquid phase 104, or just liquid 104, at the bottom. For purposes of this discussion the liquid is tetracontane having a melting point of about 80° C. to 84° C. and a normal boiling point of about 524° C. For purposes of discussion, the reaction zone is maintained with a head pressure of about 200 kPa absolute. The reactor 102 can be jacketed with heating coils such as high-pressure steam or electrical resistance which can be used to at least melt the tetracontane. As shown, line 106 continuously withdraws tetracontane, passes it through furnace 108 and returns the heated tetracontane to the reactor 102. For purposes of discussion, the liquid in the reactor 102 is maintained at substantially 500° C., the tetracontane from furnace 108 is at a temperature of about 525° C. and the rate of tetracontane flow in line 106 is sufficient to provide liquid 104 at a bulk temperature of 500° C. The tetracontane in line 106 is preferably introduced at multiple points in the reactor 102 to assist in achieving a more uniform liquid temperature.

A feed of ethylene glycol diacetate is passed via line 110 to indirect heat exchanger 112 where it is vaporized and heated to a temperature below that where appreciable thermal cracking of the ethylene glycol diacetate would occur, typically in the range of about 300° C. to 400° C. As shown, the heat exchange is with tetracontane withdrawn from reactor 102 via line 114 and passed to heat exchanger 112. As shown, line 114 withdraws liquid from an upper region of the reactor to minimize the concentration of vinyl acetate, acetic acid and unreacted ethylene glycol diacetate in the liquid.

The vaporized and heated ethylene glycol diacetate is passed from heat exchanger 112 via line 116 to venturi mixer 118. The cooler tetracontane from heat exchanger 112 is passed via line 120 to venturi mixer 118 where it is used as a motive fluid. In venturi mixer 118, the vaporous phase containing ethylene glycol diacetate is drawn into the motive fluid as a finely divided dispersion and this mixed phase fluid is passed to nozzle 122 and introduced into a lower portion of liquid 104. Venturi mixer 118 is thus part of a vapor injection system to provide finely dispersed bubbles. With small bubbles, the bubbles tend to follow the flow of the liquid rather than taking a direct route upwardly. Often, the bubbles are less than 20 microns in diameter. Many types of vapor injectors are known and used industrially. One preferred type of vapor injector is described in U.S. Pat. No. 4,162,970. A plurality of vapor injectors can be used to distribute ethylene glycol diacetate over the cross-section of liquid 104. The injection of the motive fluid and the vaporous ethylene glycol diacetate results in agitation of liquid 104. Hence mechanical stirring of liquid 104 typically is not used. Where the bubbles are sufficiently fine that they tend to follow the flow of the liquid, agitation can be beneficial to increase residence time of the ethylene glycol diacetate in the reaction zone.

The depth of liquid 104 together with the rate of upwardly passing bubbles define the average residence time of the vaporous phase in the reaction zone. Thus, the operator can use the depth of liquid 104 to control the duration of the cracking reaction. The vapor in the head space above the surface of liquid 104 will contain vinyl acetate, acetic acid, unreacted ethylene glycol diacetate and reaction side products. This vaporous phase is withdrawn via line 124 and chilled in heat exchanger 126, e.g., with water supplied via line 128, to a temperature below that which thermally cracks ethylene glycol diacetate, e.g., below 350° C., but preferably sufficiently high to maintain the moieties in the stream in the vapor phase. The chilled vaporous phase is passed via line 130 to rectification column 132 where an overhead of vinyl acetate monomer, acetic acid and other lights is withdrawn via line 134 for further purification of the vinyl acetate monomer. A bottoms fraction containing ethylene glycol diacetate is withdrawn via line 136 and is passed to heat exchanger 112 for recycle.

A purge stream 138 is taken from line 114 to avoid the build up of undesired high boiling contaminants, such as polyvinyl acetate, in liquid 104. A purge stream 140 is taken from line 136 to avoid the build-up of contaminants in the ethylene glycol diacetate fraction.

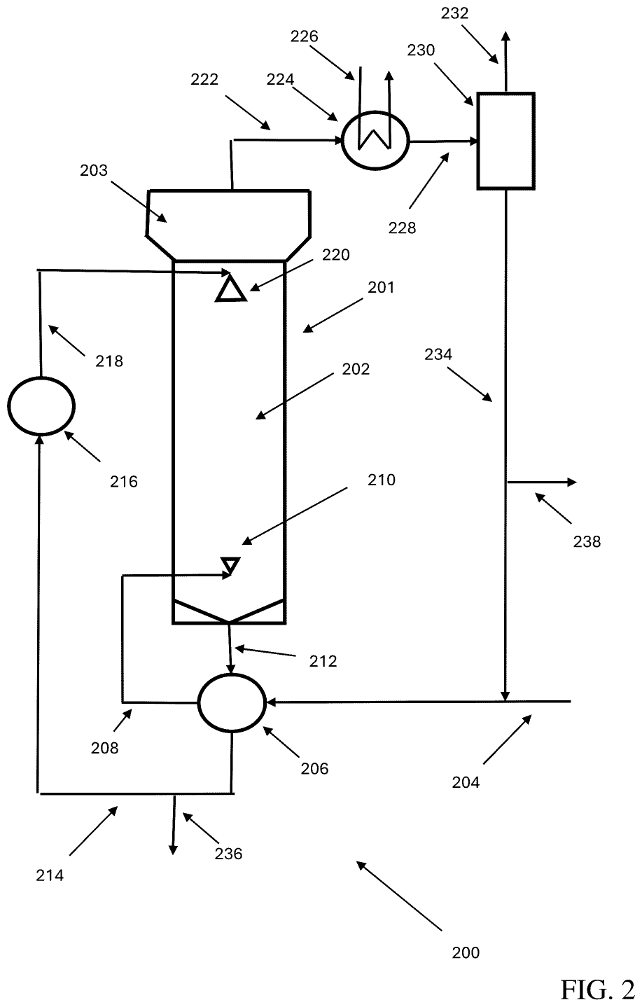

With respect to FIG. 2, a schematic depiction of apparatus 200 in which the non-reactive liquid is in the discontinuous phase is presented. Apparatus 200 comprises reactor 201 having contact zone 202 and, at the top, disentrainment zone 203. An ethylene glycol diacetate-containing feed is supplied to apparatus 200 via line 204. The feed is heated and vaporized in heat exchanger 206, which for purposes of discussion is 400° C. The vaporized feed is passed from heat exchanger 206 via line 208 to distribution nozzle 210 positioned in a lower portion of contact zone 202. The vaporous phase passes upwardly in the contact zone and is contacted with droplets of the non-reactive liquid which passes counter currently. The liquid at the bottom of contact zone 202 is withdrawn from reactor 201 via line 212 and is used for heating the incoming feed in heat exchanger 206. The liquid is then passed via line 214 to furnace 216 where it is heated to, say, 525° C. The heated liquid then passes via line 218 to distributor 220 positioned in an upper portion of contact zone 202. Distributor 220 may be of any suitable design. Typically, the droplet size is less than 10, preferably less than 2, millimeters in diameter. For purposes of discussion, the droplet size is about 1 millimeter in diameter. The amount of liquid used is sufficient to heat the incoming feed to about 500° C. Distributor 220 provides a relatively uniform distribution of liquid droplets over the cross section of contact zone 202. The rate of supply of the liquid is such that at any given time, the volume of contact zone 202 that contains liquid is about 5 percent. The distribution of the droplets taken together with their rate of descent in contact zone 202 provides liquid proximate to ethylene glycol diacetate molecules undergoing cracking and thereby attenuates heat loss proximate to the endothermic reaction. The duration of the feed in contact zone 202 is sufficient to provide the conversion of a portion of the ethylene glycol diacetate, say, between about 15 and 35 percent.

The vaporous phase exits contact zone 202 and enters disentrainment zone 203. Disentrainment zone 203 has a larger cross-sectional area and the drop in vapor velocity allows entrained liquid to fall out. At least a portion of the liquid is directed by the disentrainment zone to the sides of contact zone 202, thereby limiting contact of the ethylene glycol diacetate and reaction products with the walls. While the walls of contact zone 202 may be heated, it usually is not necessary as the heat is provided by the non-reactive liquid.

A vaporous phase is withdrawn from an upper portion of disentrainment zone 203 via line 232 and passed to heat exchanger 224 where it is chilled, e.g., with water supplied via line 226, to a temperature below that which thermally cracks ethylene glycol diacetate, e.g., below 350° C., but preferably sufficiently high to maintain the moieties in the stream in the vaporous phase. The chilled vaporous phase is passed via line 228 to rectification column 230 where an overhead of vinyl acetate monomer, acetic acid and other lights is withdrawn via line 232 for further purification of the vinyl acetate monomer. A bottoms fraction containing ethylene glycol diacetate is withdrawn via line 234 and is passed to heat exchanger 206 for recycle.

A purge stream 236 is taken from line 214 to avoid the buildup of undesired high boiling contaminants, such as polyvinyl acetate, in the liquid. A purge stream 238 is taken from line 234 to avoid the build-up of contaminants in the ethylene glycol diacetate fraction.

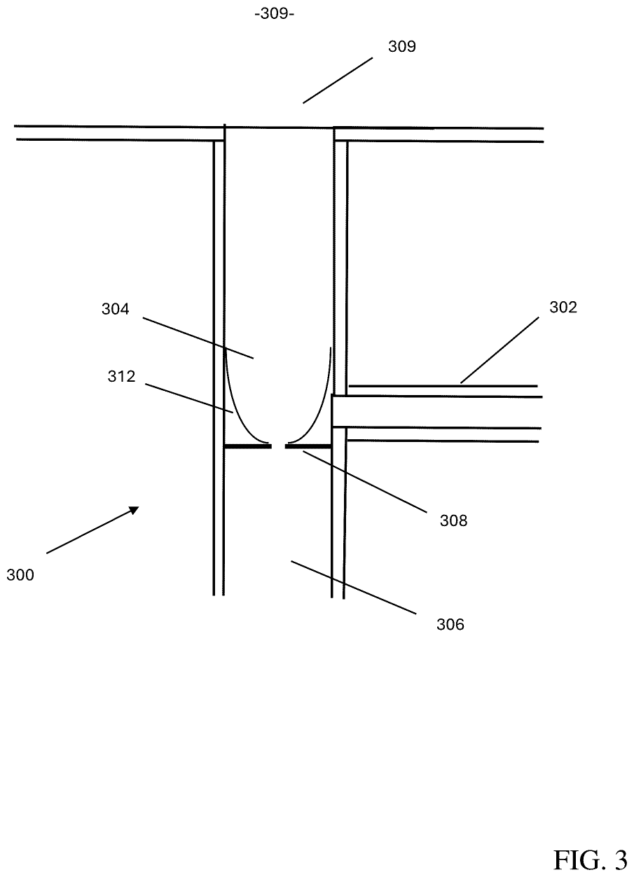

With respect to FIG. 3 an injector 300 is depicted for the admixing of ethylene glycol diacetate feed and non-reactive liquid. The injector 300 can serve as all or a part of the reaction zone. The ethylene glycol diacetate feed is passed through line 302 to a throat region 304 of the injector. Hot liquid, e.g., at 550° C., is passed via line 306 into injector 300 and passes through an orifice plate 308 into throat region 304. As shown, when the hot liquid passes through orifice plate 308, a low-pressure region 312 is formed which assists in the admixing of the ethylene glycol feed and the hot liquid. If the feed is a liquid, it is vaporized and forms a two-phase stream upon vaporization of the feed in which the hot liquid is the continuous phase. If the feed is vaporous, the low pressure facilitates the inclusion of the feed as fine bubbles in the hot liquid.

For purposes of discussion, the two-phase mixture is at a temperature of about 500° C. which enables cracking of the ethylene glycol diacetate to vinyl acetate and acetic acid. Throat region 304 may be sufficient in length to enable a desired degree of conversion, i.e., the throat region serves in essence as a tubular reactor. In this case, the throat region 304 directs the mixture to a disentrainment zone 309 within the reactor vessel 310 for liquid and vapor phase separation. As discussed before, the liquid can be heated and recycled. The vaporous phase can be processed to recover vinyl acetate and unreacted ethylene glycol diacetate, which can be recycled. Alternatively, throat region 304 can direct the mixture to a reactor vessel 310 for continuing the reaction of ethylene glycol diacetate to vinyl acetate and acetic acid. The vinyl acetate product can be obtained from the vaporous effluent from reactor vessel 310 in the manner described above in respect of FIGS. 1 and 2.

Although the disclosure has been described with reference to preferred implementations, persons skilled in the art will recognize that changes may be made in form and detail without departing from the spirit and scope of the disclosed apparatus, systems and methods.

What is claimed is:

1. A process for converting ethylene glycol diacetate to vinyl acetate comprising: continuously supplying ethylene glycol diacetate to a reaction zone, said ethylene glycol diacetate; contacting in the reaction zone the ethylene glycol diacetate with a substantially non-reactive liquid, said liquid being (i) at a temperature higher than the boiling point at the pressure in the reaction zone of the ethylene glycol diacetate supplied such that a mixed liquid-vapor phase exists in the reaction zone, and (ii) at a temperature sufficient to convert at least a portion of the ethylene glycol diacetate to vinyl acetate monomer and acetic acid and provide a product gas comprising vinyl acetate monomer, acetic acid and unreacted ethylene glycol diacetate; and continuously withdrawing said product gas from the reaction zone.

2. The process of claim 1 wherein the non-reactive liquid is provided as a continuous phase in the reaction zone.

3. The process of claim 1 wherein the non-reactive liquid is provided as a discontinuous phase in the reaction zone.

4. The process of claim 1 wherein the non-reactive liquid is at a temperature between about 450° C. and 550° C.

5. The process of claim 1 wherein the ethylene glycol diacetate supplied is vaporous.

6. The process of claim 1 wherein between about 15 and 50 percent of the ethylene glycol diacetate is converted in the reaction zone.

7. The process of claim 1 wherein the non-reactive liquid has a boiling point under the pressure in the reaction zone at least about 10° C. higher, than the temperature in the reaction zone.

8. The process of claim 1 wherein the non-reactive liquid is an aromatic or aliphatic hydrocarbon of at least 30 carbon atoms.

9. The process of claim 1 wherein the time that the vaporous phase is at a temperature of above 350° C. is less than about 50 seconds.

10. The process of claim 1 wherein non-reactive liquid is at a temperature between about 450° C. and 550° C. and the time that the vaporous phase is at a temperature of above 350° C. is less than about 10 seconds.

11. The process of claim 1 wherein the non-reactive liquid is provided as a continuous phase in the reaction zone and the vaporous phase comprises bubbles less than 5 millimeters in diameter.

12. The process of claim 11 wherein the vaporous phase bubbles are less than 1 millimeter in diameter.

13. The process of claim 1 wherein the ethylene glycol diacetate is supplied as a vapor and at a temperature between about 250° C. and 450° C.

14. The process of claim 1 wherein the ethylene glycol diacetate is supplied as a liquid and is vaporized by contact with the non-reactive liquid.
